(19) United States
(12) Patent Application Publication
(10) Pub. No.: US 2019/0374496 A1
(43) Pub. Date: Dec. 12, 2019

(54) TREATMENT WITH HIGHLY PURIFIED EICOSAPENTAENOIC ACID AS FREE FATTY ACID IMPROVES INFLAMMATION, AFFECTS COLONIC DIFFERENTIATION MARKERS AND MICROBIOTA IN PATIENTS WITH ULCERATIVE COLITIS

(71) Applicant: S.L.A. PHARMA AG, LIESTAL (CH)

(72) Inventors: ANDREA BELLUZZI, BOLOGNA (IT); LUIGI RICCIARDIELLO, BOLOGNA (IT); JUSTIN SLAGEL, HERTFORDSHIRE (GB)

(21) Appl. No.: 16/539,219

(22) Filed: Aug. 13, 2019

Related U.S. Application Data
(63) Continuation-in-part of application No. PCT/IB2018/000176, filed on Feb. 14, 2018.
(60) Provisional application No. 62/458,715, filed on Feb. 14, 2017.

(51) Int. Cl. A61K 31/202; A61K 9/48

(52) U.S. Cl. CPC A61K 31/202; A61K 9/4891

(57) ABSTRACT

This present invention relates to the use of eicosapentaenoic acid (EPA) for the treatment of ulcerative colitis (UC), and more particularly, the use of highly purified eicosapentaenoic acid as free fatty acids (EPA-FFA) having a purity of at least 95% for reducing inflammation in a subject suffering from ulcerative colitis and wherein the levels of IL-10 and SOCS3 are increased and the microbiome of the intestinal mucosal tissue is favorably modulated.

### CROSS-REFERENCE TO RELATED APPLICATION

The present application is a Continuation-in-Part application and claims priority to copending International Application No. PCT/IB2018/0176 filed on Feb. 14, 2018, which in turn claims priority U.S. Provisional Patent Application Ser. No. 62/458,715, filed on Feb. 14, 2017.

### BACKGROUND OF THE INVENTION

### Technical Field

This invention relates to the use of eicosapentaenoic acid (EPA) for the treatment of ulcerative colitis (UC), and more particularly, the use of highly purified eicosapentaenoic acid as free fatty acids (EPA-FFA) for reducing inflammation, and modifying microbiota in a subject suffering from ulcerative colitis.

### Related Art

Patients with ulcerative colitis (UC) have an increased risk to develop colitis-associated cancer (CAC) which is proportionally related to the duration and the extent of the disease (1). Current strategies to prevent CAC development are mainly based on endoscopic surveillance in order to intercept and eradicate dysplasia which can evolve to a malignant transformation (2). However, persistent active intestinal inflammation may hamper the identification of dysplastic areas during endoscopy. Thus, despite the reduction of advanced cancer incidence rates, obtained through a regular endoscopic surveillance, critical goals for CAC prevention remain to preserve a condition of histological remission (3, 4), and to have predictive markers indicating those patients in whom endoscopic surveillance would be more effective. Fecal calprotectin (FC) is a cytosolic protein belonging to the S100 protein family, abundant in neutrophil granulocytes (5), which represents a good predictor of endoscopic activity also in asymptomatic UC patients (6).

Several relevant molecular mechanisms contribute to the malignant epithelial transformation during chronic intestinal inflammation. Among these, aberrant activation of the signal transducer and activator of transcription 3 (STAT3), Interleukin (IL)-10 deficiency or impaired function are critically involved in the onset of CAC7, (8).

Moreover, a thin and penetrable mucus layer, allowing a direct contact of bacteria with the epithelium, can lead to persistent colonic inflammation, thus promoting colon cancer development in UC patients (9). Indeed, an over-growth of mucosal and fecal bacteria in inflamed colonic mucosa has been observed in UC patients, thus supporting a critical role of the intestinal microbiota in the pathogenesis of UC and progression to CAC (10, 11).

The canonical Notch signaling pathway, through the modulation of the transcriptional target hairy and enhancer of split 1 (HES1), the antagonists atonal homolog 1 (HATH1) and kruppel-like factor 4 (KLF4) target, is crucial to preserve a proper intestinal differentiation (12, 13). The abnormal regulation of these transcriptional factors results in a compromised epithelial differentiation which can lead to an inefficient control of pathogenic microbes growth, favoring a tumor-prone microenvironment (16).

The use of anti-inflammatory agents as tools for CAC prevention has been an intense focus of research (17, 18). To date, there are no uncontested chemopreventive agents for CAC. Long-standing ulcerative colitis patients are at high-risk of developing colorectal cancer (CAC). Ulcerative colitis (UC) is a chronic inflammatory condition affecting the colon, characterized by alternating periods of activity and remission. The clinical activity is always preceded by a progressive asymptomatic mucosal inflammation. Importantly, the treatment of UC may inhibit or reduce the development of cancer.

Accordingly, there is a need for a treatment that has the ability to reduce and/or treat the UC with the end result of reducing inflammation and effecting both goblet cells differentiation markers and microbiota in patients suffering from ulcerative colitis.

### SUMMARY OF THE INVENTION

This present invention relates to the use of the use of highly purified eicosapentaenoic acid as free fatty acids (EPA-FFA) having a purity of at least 95% purity and more preferably 99% purity for reducing inflammation in a subject suffering from ulcerative colitis, increasing levels of IL-10 of SOCS-3, induction of Hes-1 and KLF-4 associated with goblet cells differentiation and favorably modulating the microbiome of the intestinal mucosal tissue in such a subject.

In another aspect, the present invention provides a method of inducing and increasing levels of IL-10 and suppressor of cytokine signaling-3 (SOCS3), the method comprising administering to a subject suffering from ulcerative colitis, a therapeutic amount of eicosapentaenoic acid in the free fatty acid (EPA-FFA) form having purity of at least 95%, and more preferably at least 99%, wherein the therapeutic amount is in an amount from about 250 mg to 4 g per day, and more preferably in an amount from about 600 mg to about 2 g per day.

Notably with the increase of SOCS3, there is a committal partial inhibition of signal transducer and activator of transcription-3 (STAT3) activation.

Preferably the EPA-FFA is administered for about 45 days to at least 6 months and more preferably about 90 days.

In yet another aspect, the present invention provides a method to modulate the intestinal microbiota of the mucosal tissue in a subject suffering from ulcerative colitis, the method comprising administering to a subject a therapeutic amount of eicosapentaenoic acid in the free fatty acid (EPA-FFA) form having purity of at least 95%, and more preferably at least 99%, wherein the therapeutic amount is in an amount from about 250 mg to 4 g per day, and more preferably in an amount from about 600 mg to about 2 g per day, for a period of about 45 days to at least 6 months and more preferably about 90 days, wherein the levels of fecal Prevotellaceae and Porphyromonadaceae families are increased and the level of mucolytic Bacteroides spp at mucosal level is decreased.

In a still further aspect, the present invention provides for a method of inducing of KLF-4 to promote goblet cells differentiation, the method comprising the administering to a subject suffering from ulcerative colitis a therapeutic amount of eicosapentaenoic acid in the free fatty acid (EPA-FFA) form having purity of at least 95%, and more preferably at least 99%, wherein the therapeutic amount is in an amount from about 250 mg to 4 g per day, and more preferably in an amount from about 600 mg to about 2 g per day, for a period of preferably about 45 days to at least 6 months and more preferably about 90 days.

In another aspect, the present invention provides for use of eicosapentaenoic acid in the free fatty acid (EPA-FFA) form having purity of at least 95%, and more preferably at least 99% for the increase of levels of IL-10 or SOCS-3, induction of Hes-1 and KLF-4 associated with goblet cells differentiation and favorably modulating the microbiome of the intestinal mucosal tissue in a subject suffering from UC, the EPA-FFA is in an amount from about 250 mg to 4 g per day, and more preferably in an amount from about 600 mg to about 2 g per day, for a period of preferably about 45 days to at least 6 months and more preferably about 90 days.

These and other advantages and features of the present invention will be described more fully in a detailed description of the preferred embodiments which follows.

### DETAILED DESCRIPTION OF THE INVENTION

The present invention is based on the findings that administration of EPA-FFA is effective in vivo in reducing the development of colorectal cancer in subjects suffering of ulcerative colitis. Further, the present invention provides for a modulating of the microbiome of the intestinal track. Still further the present invention provides for induction of IL-10 and SOCS3 while partially inhibiting the activation of STAT3.

Preferably, the EPA-FFA used for making the medical preparations, medicaments or compositions in accordance with the invention is of at least about 90% purity and will contain no more than minimal or pharmaceutically insignificant amounts of any other polyunsaturated fatty acids. A purity of more than 95% is recommended with the highest commercially available grade (about 99% purity), which is substantially free of any other polyunsaturated fatty acids, being the most preferred material.

Thus, according to one aspect of the present invention, highly purified eicosapentaenoic acid as a free fatty acid is used to make a medical preparation or medicament for the treatment of malignant tumors in mammals.

EPA may be found in fish oil, plants or microorganisms as free fatty acids or in conjugated forms such as acylglycerols, phospholipids, sulfolipids or glycolipids, and may be extracted through a variety of means well-known in the art. Such means may include extraction with organic solvents, such as methanol and chloroform, sonication, supercritical fluid extraction using for example carbon dioxide, and physical means such as presses, or combinations thereof. Where desirable, the aqueous layer can be acidified to protonate negatively charged moieties and thereby increase partitioning of desired products into the organic layer. After extraction, the organic solvents can be removed by evaporation under a stream of nitrogen. When isolated in conjugated forms, the products may be enzymatically or chemically cleaved to release the free fatty acid or a less complex conjugate of interest, and can then be subject to further manipulations to produce a desired end product.

If further purification is necessary, standard methods can be employed. Such methods may include extraction, treatment with urea, fractional crystallization, HPLC, fractional distillation, silica gel chromatography, high speed centrifugation or distillation, or combinations of these techniques. Protection of reactive groups, such as the acid or alkenyl groups, may be done at any step through known techniques, for example alkylation or iodination. Protecting groups may be removed at any step. Desirably, purification of fractions containing EPA may be accomplished by initial esterfication, treatment with urea, supercritical fluid extraction and chromatography with the subsequent isolation of the free fatty acid.

In order to isolate EPA from the triglyceride it is necessary to free the fatty acids by hydrolysis or ester exchange in order that purification can be effected. Purification can be achieved by techniques such as fractional distillation, molecular distillation and chromatography. A particularly desirable chromatographic method employs super-critical fluids using, for example, carbon dioxide as the mobile phase, such as described in European Patent EP 0 712 651. It has been found that using such techniques EPA may be purified to levels approaching 100 percent. For practical reasons the EPA may be purified as its ethyl or methyl ester and hydrolyzed back to the free fatty acid form. Purification enables a product to be prepared which is highly concentrated and free from other fatty acids that are less desirable in the finished product. In addition, other chemicals entities such as mono- and di-glycerides, hydrocarbons, pesticide residues and the like can be removed. The highly purified EPA is thus suitable for human ingestion as it contains substantially reduced levels of toxins, compounds contributing to unpalatability or undesirable fatty acids such as saturated fatty acids. The free fatty acid form of EPA can be absorbed in the gut easily without need of prior enzymatic conversion. Using this method about 150 kg of unpure EPA can be converted into 50 kg of essentially pure EPA-FFA, that being at least 90% purity.

If further purification is necessary, standard methods can be employed. Such methods may include extraction, treatment with urea, fractional crystallization, HPLC, fractional distillation, silica gel chromatography, high speed centrifugation or distillation, or combinations of these techniques. Protection of reactive groups, such as the acid or alkenyl groups, may be done at any step through known techniques, for example alkylation or iodination. Protecting groups may be removed at any step. Desirably, purification of fractions containing EPA may be accomplished by initial esterfication, treatment with urea, supercritical fluid extraction and chromatography with the subsequent isolation of the free fatty acid.

A preferred EPA free fatty acid is commercially available under the tradename ALFA™ (S.L.A. Pharma, UK). This PUFA is 99% pure EPA, in a free fatty acid form and formulated into a pH-dependent, enteric-coated capsules designed to ensure release of the contents in the small intestine at pH 5.5. Other constituents include AA (≤0.5%) and trace amounts of other fatty acids. Key advantages of this preparation of EPA are its high degree of purity compared with many fish oil products, its presentation as the free fatty acid maximizing systemic bioavailability, ease of dosage in 500 mg capsules and a delayed-release profile, which minimizes gastro-intestinal side-effects.

Preferably the 99% pure EPA is administered in an amount from about 250 mg to 4 g per day and more preferably from about 600 mg to about 2 g daily. The dosage may be administered daily, weekly or longer for about 1 to 12 months. Notably, the tolerability of 99% pure EPA as ALFA™ capsules is excellent and the predominant small bowel delivery of EPA minimized any unpleasant taste and smell sensations that have previously hampered therapy with other fish oil preparations.

The EPA-FFA alone or in combination with another therapeutic agent used to treat UC may be formulated in multiple delivery modes. The active agent can be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

A solid composition form may include a solid carrier and one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredients. In tablets, the active ingredients are mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution); alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellent.

Having described the invention in detail, it will be apparent that modifications and variations are possible without departing the scope of the invention defined in the appended claims. Furthermore, it should be appreciated that the following examples are provided as non-limiting examples.

### EXAMPLES

A study will be conducted with patients having long-standing UC. Specifically, about 20 long-standing UC patients in stable clinical remission (SCCAI=0) and with fecal calprotectin (FC)>150 μg/g measured in stools are recruited and treated with 2 g/day of EPA-FFA for 90 days. Biopsies and biological samples are collected at the entry (TO) and at the end of the study (T3). Compliance is evaluated by EPA incorporation into red blood cell membranes. Protein levels of Jagged1, Hes1, STAT3, phospho-STAT3 and KLF-4 are determined by western blotting. IL-22, IL-10 and SOCS3 mRNA levels are analyzed by qRT-PCR. Goblet cells are stained by Alcian blue. Microbiota analyses are performed on fecal and biopsies DNA samples sequencing the V3-V4 region of the bacterial 16S rRNA gene. As a reference, a healthy adult population is used. Endoscopic and histologic disease activities are measured by Mayo and Geboes scores respectively.

Fatty acids composition are evaluated on RBC-purified membranes noting that EPA-FFA supplementation leads to a significant increase of EPA. Since EPA can be converted into the ω-3 PUFA docosahexaenoic acid (DHA) in vivo through docosapentaenoic acid (DPA)(19), the overall ω-3 PUFAs content is also measured including EPA, DPA and DHA, in the patients.

EPA-FFA Supplementation Induces Both IL-10 and SOCS3 Expression Reducing STAT3 Activation

To elucidate the mechanisms responsible for the protective effect of EPA-FFA in patients with long-standing UC, the modulation of the IL-10/STAT3/SOCS3 axis is investigated in compliant and responder patients. Notably, a t significant up-regulation of IL-10 and SOCS3 mRNA levels is observed associated with an increasing trend in IL-22 mRNA. Since STAT3 represents one of the major regulators of SOCS3, it is important to characterize STAT3 activation in the subjects. Treatment with EPA-FFA reduces STAT3 Tyr705 phosphorylation (p-STAT3). The results suggest that over-expression of SOCS3 following EPA-FFA supplementation, probably as a downstream effect of IL-10 induction, reduces STAT3 activation, in particular in patients with highest percentage of EPA in RBC membranes.

EPA-FFA Modulates the Gut Microbiota Composition in UC Patients

Given the critical role of intestinal microbial imbalance in the pathogenesis of UC, the fecal and mucosal microbiota compositions are also assessed in the patients. To identify the main microbiota dysbioses associated with the long-term UC disease, the fecal microbiota composition of UC patients at TO is compared to that of a group of healthy adults. In UC subject there is likely an enrichment of the families Clostridiaceae and Ruminococcaceae, and depletion of Verrucomicrobiaceae, Peptostreptococcaceae, Porphyromonadaceae in particular genus Parabacteroides. Noteworthy EPA-FFA supplementation increases Porphyromonadaceae and decreases Ruminococcaceae in feces of UC patients. In addition, EPA-FFA had also effects on mucosal microbiota of UC patients by decreasing the abundance of mucosal-adherent members of the Bacteroidaceae family.

Discussion

Different therapeutic approaches have been tested for CAC prevention in patients with inflammatory bowel disease (IBD) over the years. Importantly, an increasing number of data obtained from in vitro experiments, as well as, animal and clinical studies support a protective role for ω-3 PUFAs (EPA and DHA) in gastrointestinal cancer prevention including CRC (as reviewed by Eltweri et al. (22)).

However, data on pharmacological and natural compounds as anticancer agents in IBD patients are elusive and still inconsistent (23). It is well known that symptoms in IBD and serum biomarkers do not always properly mirror the inflammatory degree of the mucosa (24). FC is becoming the most useful non-invasive tool for monitoring the inflammatory status of the mucosa and the response to therapy, as well as for predicting clinical relapse in IBD patients (25).

In the present invention, the effects of EPA-FFA is tested on asymptomatic patients with long-standing UC in clinical remission who retained high FC levels (>150 μg/g) despite stable maintenance therapy. It was found that short-term EPA-FFA supplementation is associated with an increase of EPA and overall ω-3 PUFAs content (EPA, DPA and DHA) into RBCs, suggesting that EPA was incorporated by most of patients (17/19) and efficiently converted into DPA and DHA.

It is noted that EPA-FFA improves the inflammatory state in patients with long-standing UC. It is theorized that the results found herein could be explained, at least in part, by the free-fatty acid-highly pure formulation of EPA used in the testing methods.

Previous evidence supports a protective role for ω-3 PUFAs intake including both EPA and DHA in the prevention of CRC in different settings (26,27,28). However, data from ω-3 PUFAs supplementation in patients and murine models of UC are still controversial (29,30,31), and the impact of dietary ω-3 PUFAs supplementation for CAC prevention is poorly defined.

Given the increased content of ω-3 PUFAs in patients in this study, it is reasonable to speculate increase protective effects may be due to both EPA and DHA. Noteworthy, the ω-6 PUFAs content will not be related to EPA-FFA supplementation. This result could be explained by the unchanged dietary habits of enrolled patients during the study. Strikingly, increased ω-3 PUFAs content should be sufficient to induce a relevant protective response in UC patients, while possibly maintaining the same ω-6 PUFAs content as previously suggested (32).

In order to characterize the EPA-FFA short-term effects in long-standing UC patients, the testing is focused on the effects of EPA-FFA supplementation on IL-10/STAT3/SOCS3 signaling. The role of STAT3 during UC is actually controversial. Indeed, studies on animal models of IBD suggested both a deleterious and protective role of STAT3 hyperactivation during colitis (33, 34). Importantly, increased levels of phospho-STAT3 are detected in patients with active UC, as well as in dysplasia and cancer, while a progressive decreasing trend of SOCS3 levels was observed from low-grade dysplasia to UC-CRC (35). However, more recent evidence obtained in UC patients supported a role of SOCS3 over-expression in short-term disease relapse and mucosal inflammation impairing STAT3 activation (36, 37). A significant up-regulation of IL-10 and SOCS3 mRNA is to be found upon EPA-FFA supplementation with a reduction of STAT3 activation in most of the patients. As previously suggested by literature data, it is hypothesized that multiple post-transcriptional mechanisms may contribute to regulate SOCS3 and IL-10 proteins, thus explaining the absence of a correlation between changes in their mRNA and protein levels (38,39,40,41,42).

Notch signaling is also a key determinant for sustaining intestinal epithelial cells differentiation and turnover, for the integrity of the mucosal barrier, as well as for regulating malignant epithelial transformation in the colon (20). Evidence show possible oncogenic and tumor suppressor activities of HES1 and KLF4 in sporadic settings, respectively (43, 44). It has been previously shown in the AOM-DSS mouse model a loss of Notch1 signaling during CAC development partially counteracted by EPA-FFA supporting a tumor-suppressor role of this pathway during inflammation-induced intestinal tumorigenesis (14). Accordingly, Garg and colleagues previously demonstrated in the same animal model, that Matrix metalloproteinase-9 (MMP-9), activating Notch1 signaling and controlling p53 cascade, exerts a strong protective effect toward CAC development (45). Otherwise, in a recent in vitro work, it was observed a MMP-9-dependent activation of Notch1 signaling in CRC cells exposed to a conditioned medium (CM) containing multiple pro-inflammatory cytokines secreted by activated macrophages. The activation of MMP-9/Notch signaling was associated with increased CRC cells invasiveness, suggesting a tumor-prone role of Notch1 signaling in sporadic CRC. Interestingly, EPA-FFA pre-treatment of CM-exposed CRC cell lines led to reduced invasion through a Notch1 signaling switch off (46). These results, as recently reviewed (47), clearly indicate that the cell response to Notch signaling activation is not univocal resulting in oncogenic or tumor-suppressive mechanisms depending on the specific pathological context.

EPA-FFA Modulated Intestinal Differentiation Inducing Both HES1 and KLF4 Proteins and Increasing the Number of Goblet Cells.

Patients with UC in remission are generally characterized by an intact mucus layer, although a defective and penetrable intestinal barrier could be retained in some cases (48). KLF4 has a crucial role on both maturation and differentiation of goblet cells in the colon (49), and a critical role for IL-10 in the regulation of goblet cells activation during inflammation has been also previously described (50). Moreover, microbiota analysis performed in the present testing shows that the gut microbiota population constituents present in the UC group at TO were partly modulated by the EPA-FFA treatment. Indeed, the Porphyromonadaceae genus Parabacteroides, known to be decreased in UC (51), is found to increase in EPA-FFA administering. Also an increase in Prevotellaceae is shown. Also, EPA-FFA shows the capability to reduce the fecal amount of Clostridium spp. Interestingly, these proteolytic microorganisms were known to induce mucolytic metabolism in other species, i.e. Bacteroides (52). Noteworthy, mucosal-adherent members of the Bacteroides genus, known to include mucolytic species, were found to be decreased after EPA-FFA treatment, possibly contributing to the protection of the epithelium. Thus, it is theorized that the ability of EPA-FFA treatment to promote goblet cells population could be a result of multiple mechanisms including the induction of KLF-4 and IL-10, as well as the reduction of mucolytic bacteria.

In conclusion, use of EPA-FFA improves endoscopic and histological inflammation, affects the IL-10/STAT3/SOCS3 cascade, stimulates goblet cells differentiation and modulates the long-term UC-related colonic alterations of intestinal microbiota.

Methods

Study Design

Eligible patients are asymptomatic subjects aged 18-70 years with long-standing (≥8 years) UC, in stable clinical remission (simple clinical colitis activity index; SCCAI=0), and FC levels higher than 150 μg/g (53). Exclusion criteria are (1) recent use of steroids (<2 months) or other experimental drugs (<3 months); (2) concomitant use of anticoagulants; (3) probiotic use; (4) pregnancy or breast-feeding; (5) known or suspected hypersensitivity to eicosapentaenoic acid or ω-3 PUFAs; and (6) severe co-morbidities. Subjects are given oral supplementation of 2 g/daily (two 500 mg capsules twice a day) of EPA-FFA (ALFA™, SLA Pharma AG, Switzerland) for 90 days. During the study, subjects are asked to keep their dietary habits. Patients undergo endoscopic examination at enrollment and after 90 days of EPA-FFA supplementation. Biopsies are taken from the sigmoid colon at each time point. Blood samples are obtained for isolation of peripheral erythrocytes. Adherence to EPA-FFA supplementation is evaluated both by capsule counting and assessing EPA incorporation into red blood cell (RBC) membranes. Compliant patients are considered those who consumed at least 80% of the capsules, without interruption of the protocol for more than 14 consecutive days.

Fecal Calprotectin Dosage

Fecal samples are collected within 24 hours before endoscopy and stored at 2-8° C. until assaying. Quantification of FC is carried out using CalFast (Eurospital, Trieste, Italy) according to the manufacturer's protocol. FC values >150 μg/g are considered predictive of mucosal endoscopic activity as previously demonstrated (53).

Endoscopic and Histological Evaluation

Two investigators will perform all endoscopies. According to the Mayo endoscopic sub-score, a cut-off ≥1 is used to discriminate the presence of endoscopic inflammation (54). Histological activity is assessed by one expert blinded pathologist and scored according to the Geboes grading system (55). A Geboes cut-off score ≥3.1 is assumed to define active histological inflammation (56). When biopsies showed different degrees of activity, the highest degree of inflammation is considered.

Acidic Mucins Quantification

Formalin-fixed and paraffin-embedded (FFPE) biopsies are de-waxed in toluene for 10 minutes, rehydrated, placed in the Alcian blue solution (Alcian blue 8GX in 3% acetic acid solution pH 2.5) for 30 minutes and counterstained with hematoxylin. For analysis, slides are placed in order of increasing Alcian blue staining intensity using a rank order scoring system (1=lower rank; 36=higher rank). Rank ordering method has been shown to be better than categorical scoring system to identify subtle differences between groups (57).

Immunoistochemistry

Immunohistochemistry (IHC) is performed on FFPE colonic sections. Slides are dewaxed, subjected to endogenous peroxidase inhibition, rehydrated and treated with citrate buffer (pH 6.0) at 120° C. for 15 minutes for antigen retrieval. Then, slides are incubated overnight at +4° C. with the monoclonal antibodies against Ki-67 and MUC2 (Table 1). After incubation with secondary antibody Rabbit/Mouse (1:1000, DAKO EnVision™ System Peroxidase), the signal is detected with diaminobenzidine (DAB) (Sigma-Aldrich, Saint Louis, Mo., USA). Percentages of Ki67 positive nuclei and MUC2 positive DAB areas are quantified using ImageJ software (NIH, Bethesda, Md., USA).

Membrane Fatty Acid Analysis

Membrane fatty acids content is measured in RBCs. Lipids extraction from RBC membranes, phospholipids separation and sample preparation are performed as previously described (58). Extracted fatty acid methyl-esters are then analyzed by gas-chromatography mass-spectrometry (GC-MS). Fatty acid levels are expressed as relative percentages of total fatty acids.

Western Blotting

Total protein lysates are isolated from biopsies by sonication in RIPA buffer. Forty μg of proteins for each sample are separated on a 4-12% NuPAGE Novex Bis-Tris Gels (Invitrogen™ Thermo Fisher Scientific, Waltham, Mass., USA) in MOPS buffer (Novex™, Thermo Fisher Scientific) and transferred onto nitrocellulose membrane. After blocking, membranes are incubated overnight at +4° C. with primary antibodies against HES1, KLF4, phosphorylated STAT3 (Y705), STAT3, IL-10, SOCS3 and GAPDH (Table 1). After incubation with appropriate secondary Horse-Radish-Peroxidase (HRP) conjugated antibodies (GE Healthcare Life Sciences, Little Chalfont, United Kingdom), the signal is detected with a luminol enhancer solution (WESTAR EtaC, Cyanagen, Bologna, Italy) and images are acquired using the Chemidoc™ XRS+(Biorad, Hercules, Calif., USA). Densitometric analysis is performed using Image Lab™ software.

Gene Expression Analysis

Total RNA is extracted from biopsies using Trizol® (Ambion, Thermo Fisher Scientific). One μg of total RNA is converted to cDNA using the High-Capacity RNA-to-cDNA™ Kit (Applied Biosystems™, Thermo Fisher Scientific) according to the manufacturer's instructions. qRT-PCR reactions are performed in duplicate on a MX3000p QPCR thermal cycler (Stratagene, San Diego, Calif., USA) using the SYBR® Select Master Mix for CFX (Applied Biosystems™ Thermo Fisher Scientific) and the specific primers for IL-10, IL-22, LGRS, C-MYC, MUC2, HES1 and KLF4. The primers sequences are listed on Table 2 (SEQ ID NOs: 1 to 16). mRNA expressions of SOCS3 and STAT3 are analyzed using a 5′ nuclease probe (Assay ID: Hs.PT.58.4303529; Integrated DNA Technologies, Coralville, Iowa, USA) and the Taqman® gene expression assay (Hs00374280_m1; Thermo Fisher Scientific), respectively. Fold induction levels are obtained using the 2−ΔΔCt method by normalizing against the reference gene RPS9.

Microbiota Analysis

Fecal samples are collected prior to the endoscopic preparation while mucosal samples were taken during endoscopy. Total bacterial DNA is extracted from feces using QIAamp DNA Stool Mini Kit (QIAGEN, Hilden, Germany) and from biopsies using DNeasy Blood & Tissue Mini Kit (Qiagen). For all samples the V3-V4 region of the bacterial 16S rRNA gene is amplified and sequenced using the Illumina platform (Illumina, San Diego, Calif.) using a 2×300 bp paired-end protocol. Indexed libraries are pooled at equimolar concentrations, denatured and diluted to 6 pmol/L before loading onto the MiSeq flow cell. Raw sequences are processed using a pipeline combining PANDAseq and QIIME. Relative abundance profiles at family or genus level are obtained and plotted. For fecal microbiota analysis, a comparison with a control population of healthy adults is also performed (21).

Statistical Analysis

Data are analyzed with Graphpad 5.0 Software (GraphPad Software Inc., CA, USA) and Statistix 9.0. Sign test, a test for analyzing simple +/− differences between paired comparisons (59), is used to analyze differences in the Mayo sub-score and Geboes score. Correlation analyses are carried out using Spearman's correlation coefficient (rs). For qRT-PCR and western blot analyses data are presented upon square-root transformation. For microbiota analysis, median differences among groups are tested using a non-parametric approach (Mann-Whitney U test); P values are corrected for multiple comparisons using the Benjamini-Hochberg method. P values <0.05 are considered statistically significant.

### REFERENCES

The contents of the references cited herein are incorporated by reference herein for all purposes.
1. Beaugerie, L. & Itzkowitz, S. H. Cancers Complicating Inflammatory Bowel Disease. N. Engl. J. Med. 373, 195 (2015).
2. Cairns, S. R. et al. Guidelines for colorectal cancer screening and surveillance in moderate and high risk groups (update from 2002). Gut 59, 666-89 (2010).
3. Choi, C.-H. R. et al. Forty-Year Analysis of Colonoscopic Surveillance Program for Neoplasia in Ulcerative Colitis: An Updated Overview. Am. J. Gastroenterol. 110, 1022-1034 (2015).
4. Pineton de Chambrun, G., Peyrin-Biroulet, L., Lémann, M. & Colombel, J.-F. Clinical implications of mucosal healing for the management of IBD. Nat. Rev. Gastroenterol. Hepatol. 7, 15-29 (2010).
5. Poullis, A., Foster, R., Mendall, M. A. & Fagerhol, M. K. Emerging role of calprotectin in gastroenterology. J. Gastroenterol. Hepatol. 18, 756-62 (2003).
6. Mooiweer, E., Fidder, H. H., Siersema, P. D., Laheij, R. J. F. & Oldenburg, B. Fecal calprotectin testing can identify ineffective colorectal cancer surveillance procedures in patients with longstanding colitis. Inflamm. Bowel Dis. 20, 1079-84 (2014).
7. Grivennikov, S. et al. IL-6 and Stat3 Are Required for Survival of Intestinal Epithelial Cells and Development of Colitis-Associated Cancer. Cancer Cell 15, 103-113 (2009).
8. Sturlan, S. et al. Interleukin-10-deficient mice and inflammatory bowel disease associated cancer development. Carcinogenesis 22, 665-71 (2001).
9. Johansson, M. E. V. et al. Bacteria penetrate the normally impenetrable inner colon mucus layer in both murine colitis models and patients with ulcerative colitis. Gut 63, 281-291 (2014).
10. Chu, H. et al. Gene-microbiota interactions contribute to the pathogenesis of inflammatory bowel disease. Science 352, 1116-20 (2016).
11. Tjalsma, H., Boleij, A., Marchesi, J. R. & Dutilh, B. E. A bacterial driver-passenger model for colorectal cancer: beyond the usual suspects. Nat. Rev. Microbiol. 10, 575-82 (2012).
12. Neurath, M. F. New targets for mucosal healing and therapy in inflammatory bowel diseases. Mucosal Immunol. 7, 6-19 (2014).
13. Katz, J. P. et al. The zinc-finger transcription factor Klf4 is required for terminal differentiation of goblet cells in the colon. Development 129, 2619-28 (2002).
14. Piazzi, G. et al. Eicosapentaenoic acid free fatty acid prevents and suppresses colonic neoplasia in colitis-associated colorectal cancer acting on Notch signaling and gut microbiota. Int. J. cancer 135, 2004-13 (2014).
15. Ghaleb, A. M., Laroui, H., Merlin, D. & Yang, V. W. Genetic deletion of Klf4 in the mouse intestinal epithelium ameliorates dextran sodium sulfate-induced colitis by modulating the NF-κB pathway inflammatory response. Inflamm. Bowel Dis. 20, 811-20 (2014).
16. Sartor, R. B. & Mazmanian, S. K. Intestinal Microbes in Inflammatory Bowel Diseases. Am. J. Gastroenterol. 1(Suppl.), 15-21 (2012).
17. Terdiman, J. P., Steinbuch, M., Blumentals, W. A., Ullman, T. A. & Rubin, D. T. 5-Aminosalicylic acid therapy and the risk of colorectal cancer among patients with inflammatory bowel disease. Inflamm. Bowel Dis. 13, 367-71 (2007).
18. Lasry, A., Zinger, A. & Ben-Neriah, Y. Inflammatory networks underlying colorectal cancer. Nat. Immunol. 17, 230-40 (2016).
19. Arterburn, L. M., Hall, E. B. & Oken, H. Distribution, interconversion, and dose response of n-3 fatty acids in humans. Am. J. Clin. Nutr. 83, 1467S-1476S (2006).
20. Obata, Y. et al. Epithelial cell-intrinsic Notch signaling plays an essential role in the maintenance of gut immune homeostasis. J. Immunol. 188, 2427-36 (2012).
21. Biagi, E. et al. Gut Microbiota and Extreme Longevity. Curr. Biol. 26, 1480-5 (2016).
22. Eltweri, A. M. et al. Potential applications of fish oils rich in omega-3 polyunsaturated fatty acids in the management of gastrointestinal cancer. Clin. Nutr. 36, 65-78 (2017).
23. Subramanian, V. & Logan, R. F. Chemoprevention of colorectal cancer in inflammatory bowel disease. Best Pract. Res. Clin. Gastroenterol. 25, 593-606 (2011).
24. Vermeire, S., Van Assche, G. & Rutgeerts, P. Laboratory markers in IBD: useful, magic, or unnecessary toys? Gut 55, 426-31 (2006).
25. Sandborn, W. J. et al. Correlation Between Concentrations of Fecal Calprotectin and Outcomes of Patients With Ulcerative Colitis in a Phase 2 Trial. Gastroenterology 150, 96-102 (2016).
26. Song, M. et al. Marine ω-3 polyunsaturated fatty acids and risk of colorectal cancer according to microsatellite instability. J. Natl. Cancer Inst. 107 (2015).
27. Fini, L. et al. Highly purified eicosapentaenoic acid as free fatty acids strongly suppresses polyps in Apc(Min/+) mice. Clin. Cancer Res. 16, 5703-11 (2010).
28. Cockbain, A. J., Toogood, G. J. & Hull, M. A. Omega-3 polyunsaturated fatty acids for the treatment and prevention of colorectal cancer. Gut 61, 135-149 (2012).
29. Matsunaga, H. et al. Omega-3 fatty acids exacerbate DSS-induced colitis through decreased adiponectin in colonic subepithelial myofibroblasts. Inflamm. Bowel Dis. 14, 1348-57 (2008).
30. Jia, Q. et al. Reduced colitis-associated colon cancer in Fat-1 (n-3 fatty acid desaturase) transgenic mice. Cancer Res. 68, 3985-91 (2008).
31. Ananthakrishnan, A. N. et al. Long-term intake of dietary fat and risk of ulcerative colitis and Crohn's disease. Gut 63, 776-84 (2014).
32. Davidson, L. A. et al. Chemopreventive n-3 polyunsaturated fatty acids reprogram genetic signatures during colon cancer initiation and progression in the rat. Cancer Res. 64, 6797-804 (2004).
33. Suzuki, A. et al. CIS3/SOCS3/SSI3 plays a negative regulatory role in STAT3 activation and intestinal inflammation. J. Exp. Med. 193, 471-81 (2001).
34. Pickert, G. et al. STAT3 links IL-22 signaling in intestinal epithelial cells to mucosal wound healing. J. Exp. Med. 206, 1465-72 (2009).
35. Li, Y. et al. Disease-related expression of the IL6/STAT3/SOCS3 signalling pathway in ulcerative colitis and ulcerative colitis-related carcinogenesis. Gut 59, 227-35 (2010).
36. Xu, A. T. et al. High suppressor of cytokine signaling-3 expression impairs STAT3-dependent protective effects of interleukin-22 in ulcerative colitis in remission. Inflamm. Bowel Dis. 21, 241-50 (2015).
37. Li, Y. et al. Increased suppressor of cytokine signaling-3 expression predicts mucosal relapse in ulcerative colitis. Inflamm. Bowel Dis. 19, 132-40 (2013).
38. Tone, M., Powell, M. J., Tone, Y., Thompson, S. A. & Waldmann, H. IL-10 gene expression is controlled by the transcription factors Sp1 and Sp3. J. Immunol. 165, 286-91 (2000).
39. Ma, W. et al. The p38 mitogen-activated kinase pathway regulates the human interleukin-10 promoter via the activation of Sp1 transcription factor in lipopolysaccharide-stimulated human macrophages. J. Biol. Chem. 276, 13664-74 (2001).
40. Sharma, A. et al. Posttranscriptional regulation of interleukin-10 expression by hsa-miR-106a. Proc. Natl. Acad. Sci. USA 106, 5761-6 (2009).
41. Haan, S. et al. Tyrosine phosphorylation disrupts elongin interaction and accelerates SOCS3 degradation. J. Biol. Chem. 278, 31972-9 (2003).
42. Sasaki, A. et al. The N-terminal truncated isoform of SOCS3 translated from an alternative initiation AUG codon under stress conditions is stable due to the lack of a major ubiquitination site, Lys-6. J. Biol. Chem. 278, 2432-6 (2003).
43. Reedijk, M. et al. Activation of Notch signaling in human colon adenocarcinoma. Int. J. Oncol. 33, 1223-9 (2008).
44. Zhao, W. et al. Identification of Krüppel-like factor 4 as a potential tumor suppressor gene in colorectal cancer. Oncogene 23, 395-402 (2004).
45. Garg, P. et al. Notch1 regulates the effects of matrix metalloproteinase-9 on colitis-associated cancer in mice. Gastroenterology 141, 1381-92 (2011).
46. Fazio, C. et al. Inflammation increases NOTCH1 activity via MMP9 and is counteracted by Eicosapentaenoic Acid-free fatty acid in colon cancer cells. Sci. Rep. 6, 20670 (2016).
47. Fazio, C. & Ricciardiello, L. Inflammation and Notch signaling: a crosstalk with opposite effects on tumorigenesis. Cell Death Dis. 7, e2515 (2016).
48. Johansson, M. E. V. et al. Bacteria penetrate the normally impenetrable inner colon mucus layer in both murine colitis models and patients with ulcerative colitis. Gut 63, 281-91 (2014).
49. Ghaleb, A. M., McConnell, B. B., Kaestner, K. H. & Yang, V. W. Altered intestinal epithelial homeostasis in mice with intestine-specific deletion of the Krüppel-like factor 4 gene. Dev. Biol. 349, 310-320 (2011).
50. Hasnain, S. Z. et al. IL-10 Promotes Production of Intestinal Mucus by Suppressing Protein Misfolding and Endoplasmic Reticulum Stress in Goblet Cells. Gastroenterology 144, 357-368.e9 (2013).
51. Rajilić-Stojanović, M., Shanahan, F., Guarner, F. & de Vos, W. M. Phylogenetic analysis of dysbiosis in ulcerative colitis during remission. Inflamm. Bowel Dis. 19, 481-8 (2013).
52. Fischbach, M. A. & Sonnenburg, J. L. Eating for two: how metabolism establishes interspecies interactions in the gut. Cell Host Microbe 10, 336-47 (2011).
53. Scaioli, E. et al. Clinical application of faecal calprotectin in ulcerative colitis patients. Eur. J. Gastroenterol. Hepatol. 27, 1418-24 (2015).
54. Schroeder, K. W., Tremaine, W. J. & Ilstrup, D. M. Coated oral 5-aminosalicylic acid therapy for mildly to moderately active ulcerative colitis. A randomized study. N. Engl. J. Med. 317, 1625-9 (1987).
55. Geboes, K. et al. A reproducible grading scale for histological assessment of inflammation in ulcerative colitis. Gut 47, 404-9 (2000).
56. Mooiweer, E. et al. Low fecal calprotectin predicts sustained clinical remission in inflammatory bowel disease patients: a plea for deep remission. J. Crohns. Colitis 9, 50-5 (2015).
57. Holland, T. The comparative power of the discriminant methods used in toxicological pathology. Toxicol. Pathol. 33, 490-4 (2005).
58. Scaioli, E. et al. The Pharmacokinetic Profile of a New Gastroresistant Capsule Preparation of Eicosapentaenoic Acid as the Free Fatty Acid. Biomed Res. Int. 2015, 1-8 (2015).
59. Snedecor, G. & Cochran, W. Statistical Methods. (Iowa State University Press, 1989).

TABLE 1
List of primary antibodies used for IHC and western blot analyses

| Primary | | | | | |
|---|---|---|---|---|---|
| Antibodies | Supplier | Catalog NO. | Species | Type | Dilution |
| Ki-67 | Dako | M7240 | Mouse | Monoclonal | 1:100 |
| MUC2 | Abcam | ab134119 | Rabbit | Monoclonal | 1:500 |
| HES1 | Pierce ™ | PA5-28802 | Rabbit | Polyclonal | 1:3,000 |
| KLF4 | Pierce ™ | PA5-35303 | Rabbit | Polyclonal | 1:1,000 |
| p-STAT3 | Cell | #9145 | Rabbit | Monoclonal | 1:1,000 |
| (Y705) | Signaling | | | | |
| STAT3 | Cell | #4904 | Rabbit | Monoclonal | 1:1,000 |
| | Signaling | | | | |
| SOCS3 | Abcam | ab16030 | Rabbit | Polyclonal | 1:500 |
| IL10 | Abcam | ab34843 | Rabbit | Polyclonal | 1:500 |
| GAPDH | Abcam | ab9485 | Rabbit | Polyclonal | 1:2,000 |

TABLE 2
Primer sequences used for qRT-PCR

| | | | | | Product |
|---|---|---|---|---|---|
| Gene | Forward Primer | SEQ. | Reverse primer | SEQ. | Size (bp) |
| C-MYC | CGTAGTTGTGCTGATGTGTGG | 1 | CTCGGATTCTCTGCTCTCCTC | 2 | 272 |
| HES1 | TTGGAGGCTACGAGGTGGTA | 3 | GCCCCGTTGGGAATGAG | 4 | 64 |
| IL-10 | AAGACCCAGACATCAAGGCG | 5 | CACGGCCTTGCTCTTGTTTT | 6 | 116 |
| IL-22 | TGAATAACTAACCCCCTTTCCCTG | 7 | TGGCTTCCCATCTTCCTTTTG | 8 | 87 |
| KLF4 | CGAAGCCACACAGGTGAGAA | 9 | TACGGTAGTGCCAGGTCAGTTC | 10 | 94 |
| LGR5 | GGTGACAACAGCAGTATGGACG | 11 | GAAGGTGAACACAGCACTGAATGAA | 12 | 140 |
| MUC2 | ACCCGCTCTATGTCACCTTCC | 13 | GGGATCGCAGTGGTAGTTGT | 14 | 131 |
| RPS9 | GATTACATCCTGGGCCTGAA | 15 | ATGAAGGACGGGATGTTCAC | 16 | 161 |

What is claimed is:

1. A method of inducing and/or increasing levels of IL-10 and suppressor of cytokine signaling-3 (SOCS3) in a subject having ulcerative colitis, the method comprising administering to the subject a therapeutic amount of eicosapentaenoic acid in the free fatty acid (EPA-FFA) form having purity of at least 95%, wherein the therapeutic amount is in an amount from about 250 mg to 4 g per day.

2. The method of claim 1, wherein the purity of EPA-FFA is at least 99%.

3. The method of claim 1, wherein the therapeutic amount is in an amount 600 mg to about 2 g per day.

4. The method of claim 1, wherein the increase of SOCS3 causes a partial inhibition of signal transducer and activator of transcription-3 (STAT3) activation.

5. The method of claim 1, wherein EPA-FFA is administered for a period of about 45 days to about 6 months.

6. The method of claim 1, wherein EPA-FFA is administered for a period of about 90 days.

7. A method to modulate the intestinal microbiota of the mucosal tissue in a subject suffering from ulcerative colitis, the comprising administering to a subject a therapeutic amount of eicosapentaenoic acid in the free fatty acid (EPA-FFA) form having purity of at least 95%, wherein the therapeutic amount is in an amount from about 250 mg to 4 g per day.

8. The method of claim 7, wherein levels of fecal Prevotellaceae and Porphyromonadaceae families are increased and the level of mucolytic Bacteroides spp is decreased.

9. The method of claim 7, wherein the purity of EPA-FFA is at least 99%.

10. The method of claim 7, wherein the therapeutic amount is in an amount 600 mg to about 2 g per day.

11. The method of claim 7, wherein EPA-FFA is administered for a period of about 45 days to about 6 months.

12. The method of claim 7, wherein EPA-FFA is administered for a period of about 90 days.

13. A method of inducing of KLF-4 to promote goblet cells differentiation, the method comprising the administering to a subject suffering from ulcerative colitis a therapeutic amount of eicosapentaenoic acid in the free fatty acid (EPA-FFA) form having purity of at least 95%, wherein the therapeutic amount is in an amount from about 250 mg to 4 g per day

14. The method of claim 13, wherein the purity of EPA-FFA is at least 99%.

15. The method of claim 13, wherein the therapeutic amount is in an amount 600 mg to about 2 g per day.

16. The method of claim 13, wherein EPA-FFA is administered for a period of about 45 days to about 6 months.

17. The method of claim 13, wherein EPA-FFA is administered for a period of about 90 days.

18. Use of eicosapentaenoic acid in the free fatty acid (EPA-FFA) form having purity of at least 95% for increasing levels of IL-10 or SOCS-3, induction of Hes-1 and KLF-4 associated with goblet cells differentiation and favorably modulating the microbiome of the intestinal mucosal tissue in a subject suffering from UC, wherein the the EPA-FFA is in an amount from about 250 mg to 4 g per dosage.

19. The use of claim 18, wherein EPA-FFA is administered for a period of about 45 days to about 6 months.

20. The use of claim 18, wherein EPA-FFA is administered for a period of about 90 days.

21. The use of claim 18, wherein the EPA-FFA is in an amount from about 600 mg to about 2 g per dosage.

22. Use of a 99% pure EPA, in a free fatty acid form in a medicament for the treatment of ulcerative colitis (UC) in a subject, wherein the pure EPA is formulated into a pH-dependent, enteric-coated capsules for release of the contents in the small intestine at about pH 5.5.


SEQUENCE LISTING (16 sequences)

SEQ ID NO: 1 - DNA, 21 bp, Artificial Sequence
  Synthetic construct
cgtagttgtgctgatgtgtgg

SEQ ID NO: 2 - DNA, 21 bp, Artificial Sequence
  Synthetic Construct
ctcggattctctgctctcctc

SEQ ID NO: 3 - DNA, 20 bp, Artificial Sequence
  Synthetic Construct
ttggaggctacgaggtggta

SEQ ID NO: 4 - DNA, 17 bp, Artificial Sequence
  Synthetic Construct
gccccgttgggaatgag

SEQ ID NO: 5 - DNA, 20 bp, Artificial Sequence
  Synthetic Construct
aagacccagacatcaaggcg

SEQ ID NO: 6 - DNA, 20 bp, Artificial Sequence
  Synthetic Construct
cacggccttgctcttgtttt

SEQ ID NO: 7 - DNA, 24 bp, Artificial Sequence
  Synthetic Construct
tgaataactaaccccctttccctg

SEQ ID NO: 8 - DNA, 21 bp, Artificial Sequence
  Synthetic Construct
tggcttcccatcttccttttg

SEQ ID NO: 9 - DNA, 20 bp, Artificial Sequence
  Synthetic Construct
cgaagccacacaggtgagaa

SEQ ID NO: 10 - DNA, 22 bp, Artificial Sequence
  Synthetic Construct
tacggtagtgccaggtcagttc

SEQ ID NO: 11 - DNA, 22 bp, Artificial Sequence
  Synthetic Construct
ggtgacaacagcagtatggacg

SEQ ID NO: 12 - DNA, 25 bp, Artificial Sequence
  Synthetic Construct
gaaggtgaacacagcactgaatgaa

SEQ ID NO: 13 - DNA, 20 bp, Artificial Sequence
  Synthetic Construct
acccgctctatgtcaccttc

SEQ ID NO: 14 - DNA, 20 bp, Artificial Sequence
  Synthetic Construct
gggatcgcagtggtagttgt

SEQ ID NO: 15 - DNA, 20 bp, Artificial Sequence
  Synthetic Construct
gattacatcctgggcctgaa

SEQ ID NO: 16 - DNA, 20 bp, Artificial Sequence
  Synthetic Construct
atgaaggacgggatgttcac